(19) United States
(12) Patent Application Publication
(10) Pub. No.: US 2025/0099412 A1
(43) Pub. Date: Mar. 27, 2025

(54) METHOD OF INHIBITING DEGRADING PROTEASE ACTIVITY IN AGING

(71) Applicant: The Regents of the University of California, Oakland, CA (US)

(72) Inventors: Geert W. Schmid-Schoenbein, Del Mar, CA (US); Frank A. DeLano, San Diego, CA (US)

(21) Appl. No.: 18/730,028

(22) PCT Filed: Jan. 18, 2023
(86) PCT No.: PCT/US2023/010998

§ 371 Date: Jul. 18, 2024

Related U.S. Application Data
(60) Provisional application No. 63/300,409, filed on Jan. 18, 2022.

(51) Int. Cl. A61K 31/195; A61P 1/00; A61P 9/00; A61P 25/00

(52) U.S. Cl. CPC A61K 31/195; A61P 1/00; A61P 9/00; A61P 25/00

(57) ABSTRACT

Provided herein are methods of reversing accumulation of a serine protease. reversing cellular damage. and/or preserving extracellular matrix in an organ of a subject comprising selecting a subject at risk of damage to the organ, and administering a therapeutically effective amount of a serine protease inhibitor. The serine protease inhibitor may be an orally administered competitive inhibitor.

### CROSS-REFERENCE TO RELATED APPLICATIONS

This application claims priority to U.S. Provisional Patent Application No. 63/300,409, filed on Jan. 18, 2022. The disclosure of the prior application is considered part of the disclosure of this application, and is incorporated herein by reference in its entirety.

### BACKGROUND

Serine proteases (e.g., trypsin) activate secondary proenzymes (e.g., members of the families of matrix metalloproteinases, kallikrein, cathepsins, and others) which participate in the tissue degradation and loss of cell function due to cleavage of the extracellular cell matrix proteins (collagen degradation) and membrane receptor cleavage (e.g., cleavage of the insulin receptor, loss of insulin receptor binding sites and “insulin resistance”). Chronic matrix metalloproteinase (MMP) inhibition has also been shown to reduce symptoms of the metabolic syndrome (e.g., doxycycline, grape seed extract, resveratrol, and others) in experimental models and short-term human trials. Multiple genes have been identified in specific aging human populations and animal models that are associated (e.g. by genome wide association studies) with longevity, however, they do not identify a general mechanism for aging in different species.

### SUMMARY

Provided herein are methods reversing accumulation of a serine protease in an organ of a subject, comprising: (a) selecting a subject having or at risk of accumulation of a serine protease in the organ; and (b) administering a therapeutically effective amount of a serine protease inhibitor, thereby reversing accumulation of the serine protease in the organ of the subject.

Additionally, provided herein are methods of reversing cellular damage in an organ of a subject, comprising: (a) selecting a subject having or at risk of cellular damage to the organ; and (b) administering a therapeutically effective amount of a serine protease inhibitor, thereby reversing cellular damage in the organ of the subject.

Additionally, provided herein are methods of preserving extracellular matrix in an organ of a subject, comprising: (a) selecting a subject having or at risk of loss of extracellular matrix in the organ; and (b) administering a therapeutically effective amount of a serine protease inhibitor, thereby preserving extracellular matrix in the organ of the subject.

In some embodiments, the subject at least 40 years old. In some embodiments, the subject at least 50 years old. In some embodiments, the subject at least 60 years old. In some embodiments, the subject is not at risk of developing shock and/or septic shock. In some embodiments, the subject does not have HIV. In some embodiments, the organ selected from the group consisting of the brain, spinal cord, heart, kidney, muscle, liver, and lung. In some embodiments, the organ selected from the group consisting of the brain, heart, and muscle. In some embodiments, the organ is the brain. In some embodiments, selecting comprises selecting a subject with a brain disease or condition. In some embodiments, the brain disease or condition is selected from the group consisting of Alzheimer's Disease, dementias including frontotemporal dementia, epilepsy or other seizure disorders, mental disorder, multiple sclerosis, Huntington's Disease, Parkinson's Disease, amyotrophic lateral sclerosis, meningitis, encephalitis, brain cancer, Crutzfeldt-Jakob disease, chronic traumatic encephalopathy, long-haul COVID associated dementia, and stroke. In some embodiments, the organ is the heart. In some embodiments, selecting a subject comprises selecting a subject with heart disease or a heart condition. In some embodiments, the heart disease or condition is selected from the group consisting of coronary heart disease, angina, unstable angina, heart failure, cardiac arrhythmias, valve disease, high blood pressure, heart arrhythmias, endocarditis, pericardial disease, and cardiomyopathy. In some embodiments, the organ is muscle. In some embodiments, selecting a subject comprises selecting a subject with muscle disease or condition. In some embodiments, the muscle disease or condition is selected from the group consisting of fibromyalgia, myositis, including polymyositis and dermatomyositis, muscular dystrophy, myasthenia gravis, amyotrophic lateral sclerosis, rhabdomyolysis, cardiomyopathy, sarcopenia, Charcot-Marie-Tooth disease, multiple sclerosis, myopathy, peripheral neuropathy, and spinal muscular atrophy. In some embodiments, the organ is the kidney. In some embodiments, selecting a subject at risk comprises selecting a subject with a kidney disease or condition. In some embodiments, the kidney disease or condition is selected from the group consisting of chronic kidney disease, diabetic kidney disease, acute kidney injury, kidney stones, kidney infections, including pyelonephritis, kidney cysts, and kidney cancer. In some embodiments, the organ is the liver. In some embodiments, selecting a subject comprises selecting a subject with a liver disease or condition. In some embodiments, the liver disease or condition is selected from the group consisting of hepatitis A. hepatitis B. hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, and nonalcoholic steatohepatitis. In some embodiments, the serine protease comprises at least one of a trypsin, a subtilisin, or combinations thereof. In some embodiments, the serine protease comprises at least one of a trypsin, an elastase, a chymotrypsin, or combinations thereof. In some embodiments, the serine protease comprises a trypsin. In some embodiments, the serine protease inhibitor is a competitive inhibitor. In some embodiments, the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin. In some embodiments, the serine protease inhibitor comprises tranexamic acid. In some embodiments, the therapeutically effective amount of the serine protease inhibitor is less than 10% of the subject's digestive enzyme activity. In some embodiments, the therapeutically effective amount of the serine protease inhibitor is less than 10 μM. In some embodiments, the therapeutically effective amount of the serine protease inhibitor is less than 5 μM. In some embodiments, the serine protease inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump. In some embodiments, the serine protease inhibitor is orally administered. In some embodiments, the serine protease inhibitor is administered by a telemetry-controlled infusion pump. In some embodiments, the serine protease inhibitor is administered as a liposome composition or as a nanoparticle encapsulation. In some embodiments, the serine protease inhibitor is administered as an eye drop. In some embodiments, the telemetry-controlled infusion pump is directed toward the organ. In some embodiments, the serine protease inhibitor is administered for more than 1 week. In some embodiments, the serine protease inhibitor is administered for more than 2 weeks. In some embodiments, the serine protease inhibitor is administered for more than 4 weeks.

Provided herein are pharmaceutical compositions for the treatment of aging or age-related conditions comprising a serine protease inhibitor. In some embodiments, the pharmaceutical composition treats an age-related condition affects an organ selected from the group consisting of the brain, spinal cord, heart, kidney, muscle, liver, and lung. In some embodiments, the age-related condition affects an organ selected from the group consisting of the brain, heart, and muscle. In some embodiments, the organ is the brain. In some embodiments, the age-related condition is selected from the group consisting of Alzheimer's Disease, dementias including frontotemporal dementia, age-related loss of neuronal function, including but not limited to memory, balance, sensation, pain, including but not limited to lower back pain, epilepsy or other seizure disorders, mental disorder, multiple sclerosis, Huntington's Disease, Parkinson's Disease, amyotrophic lateral sclerosis meningitis, encephalitis, brain cancer, and transient ischemic strokes. In some embodiments, the organ is the heart. In some embodiments, the age-related condition is selected from the group consisting of coronary heart disease, angina, unstable angina, heart failure, valve disease high blood pressure, heart arrhythmias, endocarditis, pericardial disease, and cardiomyopathy. In some embodiments, the organ is muscle. In some embodiments, the age-related condition is selected from the group consisting of fibromyalgia, myositis, including polymyositis and dermatomyositis, muscular dystrophy, myasthenia gravis, amyotrophic lateral sclerosis, rhabdomyolysis, cardiomyopathy, sarcopenia, Charcot-Marie-Tooth disease, multiple sclerosis, myopathy, peripheral neuropathy, and spinal muscular atrophy. In some embodiments, the organ is the kidney. In some embodiments, the age-related condition is selected from the group consisting of acute kidney injury, kidney stones, kidney infections, including pyelonephritis, kidney cysts, the subject being in need of renal dialysis, and kidney cancer. In some embodiments, the organ is the liver. In some embodiments, the age-related condition is selected from the group consisting of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, and nonalcoholic steatohepatitis. In some embodiments, the serine protease inhibitor is a competitive inhibitor. In some embodiments, the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin. In some embodiments, the serine protease inhibitor comprises tranexamic acid. In some embodiments, the serine protease inhibitor is administered at less than 10% of the subject's digestive enzyme activity. In some embodiments, the serine protease inhibitor is less than 10 μM. In some embodiments, the serine protease inhibitor is less than 5 μM. In some embodiments, the serine protease inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump. In some embodiments, the serine protease inhibitor is orally administered. In some embodiments, the serine protease inhibitor is administered by a telemetry-controlled infusion pump. In some embodiments, the serine protease inhibitor is administered in as a liposome composition or a nanoparticle. In some embodiments, the serine protease inhibitor is administered as an eye drop. In some embodiments, the telemetry-controlled infusion pump is directed toward the organ. In some embodiments, the serine protease inhibitor is administered for more than 1 week. In some embodiments, the serine protease inhibitor is administered for more than 2 weeks. In some embodiments, the serine protease inhibitor is administered for more than 4 weeks.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A shows images of pancreatic trypsin accumulation in rat cardiac muscle by immunohistochemistry with a monoclonal antibody to pancreatic trypsin in young (16 week), old (100 weeks), and old treated rat (at 100 weeks, blockade of pancreatic trypsin with oral tranexamic acid (TXA) for two weeks). The figure shows original bright fields and the same images after digital color extraction of the IHC label.

FIG. 1B shows images of rat cardiac muscle collagen fragmentation in young (16 week), old (100 weeks), and old treated rat (at 100 weeks, blockade of pancreatic trypsin with oral tranexamic acid for two weeks). Left panel shows bright field view, and right panel show collagen fragmentation after color extraction.

FIG. 2A shows images of pancreatic trypsin accumulation in rat brain sections by immunohistochemistry with a monoclonal antibody to pancreatic trypsin in young (16 week), old (100 week), and old treated rats (at 100 weeks, blockade of pancreatic trypsin with oral tranexamic acid for two weeks). The figure shows original bright fields and the same images after digital color extraction of the IHC label.

FIG. 2B shows images of collagen degradation in brain sections of young (16 week), old (100 week), and old-treated rats (at 100 weeks, blockade of pancreatic trypsin with oral tranexamic acid for two weeks) by labeling with peptide that attaches to fragmented collagen fibers. The figure shows original bright fields and the same images after digital color extraction of the collagen-binding peptide label.

FIG. 3A shows images of pancreatic trypsin accumulation in rat intestine by immunohistochemistry with a monoclonal antibody to pancreatic trypsin in young (16 week), old (100 weeks), and old-treated rat (at 100 weeks, blockade of pancreatic trypsin with oral tranexamic acid (TXA) for two weeks). The figure shows original bright fields and the same images after digital color extraction of the IHC label.

FIG. 3B shows images of mucin-containing mucus layer on the epithelial cells of the small intestine stained with alcian blue (pH 2.5) in young (16 week), old (100 weeks), and old-treated rat (at 100 weeks, blockade of pancreatic trypsin with oral tranexamic acid (TXA) for two weeks). The figure shows original bright field images with corresponding color extracted images below. Additionally, the top bright field images show that epithelia cells of the small intestine stained with amylase (brown) in young (16 week), old (100 week), and old-treated (100 week, blockade of pancreatic trypsin with oral tranexamic acid (TXA) for two weeks) rats. An increase in amylase in the small intestine was observed in old rats compared to young rats, and this increase was attenuated by administration of oral tranexamic acid for two weeks.

### DETAILED DESCRIPTION

The present disclosure describes methods of inhibiting a serine protease and decreasing the activity of the serine protease outside a gastrointestinal (GI) tract in a subject.

Various non-limiting aspects of these methods are described herein, and can be used in any combination without limitation. Additional aspects of various components of the methods described herein are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, a “cell” can refer to either a prokaryotic or eukaryotic cell, optionally obtained from a subject or a commercially available source.

### Aging Process

As used herein, the term “aging” refers to the process associated with becoming older. While the term refers especially to human beings, many animals, and fungi, in the broader sense, aging can also refer to single cells within an organism which have ceased dividing (cellular senescence), show reduced cell functions (response to for example growth hormones, insulin) and gene expression. In humans, aging represents the accumulation of changes over time, encompassing physical and psychological changes. For example, aging is accompanied by a loss of cell and tissue functions, clinically manifesting co-morbidities with increased susceptibility to diseases, and eventual by full organ failure. A spectrum of biological processes (e.g., cell and mitochondrial functions, stem cell proliferation and differentiation, genetic lesions, histones, DNA repair mechanisms, epigenetics, protein folding, intra- and inter-cellular signaling, and nutrient utilization) become dysregulated, unstable, and exhausted. Pathophysiological mechanisms in aging can include impaired resistance to molecular stressors, chronic low-grade inflammation, genomic instability, telomere attrition and cellular senescence, epigenetic alterations, loss of protein homeostasis (proteostasis), deregulated nutrient sensing, stem cell exhaustion, and/or altered intercellular communication. Vascular and immunological cell functions become impaired with pathological restructuring and development of age-related risk factors and diseases, while different tissues share molecular and cellular mechanisms for micro-and macrovascular pathologies in aging. Aging is also accompanied by chronic low-grade inflammation, and since the inflammatory cascade fundamentally serves tissue repair, a chronic mechanism can exist in aging that causes tissue damage. In all organs, the cells and the extracellular matrix are known to degrade, for which mechanisms have been proposed to be due to reactive oxygen species, radiation exposure, and repeat small injuries.

Aging is among the greatest known risk factors for most human diseases: of the roughly 150,000 people who die each day across the globe, about two thirds die from age-related causes. Aging is associated with changes in dynamic biological, physiological, environmental, psychological, behavioral, and social processes. As used herein, “symptoms of biological aging” can refer to common signs and symptoms of aging that can include, but are not limited to, degradation of the extracellular matrix, increased susceptibility to infection, greater risk of heat stroke or hypothermia, skin thinning and wrinkling, bones break more easily, joint changes, ranging from minor stiffness to severe arthritis, slowed and limited movement, decrease in overall energy, constipation, urinary incontinence, cognitive impairment (e.g., slowing of thought, memory, and thinking), reduced reflexes and coordination, difficulty with balance, decrease in visual acuity, diminished peripheral vision, hearing loss, whitening or graving of hair, loss of smell, and weight loss in part due to loss of muscle tissue.

Serine protease activity in organs outside the GI tract has been discovered to serve as a mechanism for chronic and gradual loss of cell and organ functions during aging (e.g., “Autodigestion”). After synthesis in the pancreas, digestive enzymes can be discharged into the small intestine where they degrade large masses of biomolecules. In the small intestine, digestive enzymes are concentrated (e.g., at sub-mM level), fully activated and relatively non-specific to facilitate breakdown of diverse polymeric food sources into lower molecular weight monomeric nutrients. Furthermore, autodigestion of one's own intestine is primarily prevented by compartmentalization of the digestive enzymes in the lumen of the intestine by the mucin/epithelial barrier, and while this barrier is always permeable to small molecular nutrients (e.g., ions, amino acids, or monosaccharides) it generally has a low permeability to larger molecules, such as pancreatic serine proteases. However, sometimes the mucin/epithelial barrier is compromised due to disease or conditions, and sometimes the mucin/epithelial barrier becomes compromised during aging, as older individuals tend to have weaker mucin/epithelial barriers than young individuals.

The present disclosure provides mechanisms for aging due to autodigestion involving serine proteases. The methods of the disclosure block serine proteases outside the gastrointestinal tract (GI) tract with minimal effect on serine protease activity inside the GI tract to ameliorate symptoms and diseases of aging due to autodigestion.

### Serine Proteases/Serine Protease Inhibitors

Serine proteases are sometimes referred to as serine endopeptidases, which as enzymes that can cleave peptide bonds in proteins. There are two main categories of serine proteases based on their structure, chymotrypsin-like (trypsin-like) and subtilisin-like. Subtilisin-like serine proteases can be found in prokaryotes and share the same catalytic mechanism as the trypsin-like serine proteases. The chymotrypsin-like/trypsin-like serine proteases contain two beta-barrel domains that converge at a catalytic site. Serine proteases are folded in such a way that they utilize a catalytic triad located in the active site of the enzyme, which consists of three amino acids, Histidine 57, Serine 195, and Aspartic acid 102. Additionally, elastase is a serine protease produced by the pancreas that catalyzes cleavage of carboxyl groups present on small hydrophobic amino acids, such as glycine, alanine, and valine. The primary role of elastase is the breakdown of elastin, a protein that imparts elasticity to connective tissue.

Serine proteases can be inhibited by serine protease inhibitors, which can include chemical inhibitors as well as proteinaceous inhibitors. In non-limiting embodiments, small molecular weight inhibitors can pass out of the small intestine and into blood, plasma, or other tissues. Sometimes serine protease inhibitors are called SERPINs. Serine protease inhibitors can include competitive inhibitors, non-competitive inhibitors, permeant inhibitors, reversible inhibitors, and irreversible inhibitors. Sometimes serine protease inhibitors block a serine protease by changing the conformational shape of the serine protease, disrupting the active site of the serine protease. Sometimes serine protease inhibitors bind to and block the active site of a serine protease.

Non-limiting examples of serine protease inhibitors include Lepirudin, Bivalirudin, Argatroban, Chymostatin, Benzamidine, Ximelagatran, Rivaroxaban, Idraparinux, Apixaban, Otamixaban, Aprotinin, Dabigatran etexilate, Edoxaban, Letaxaban, Ulinastatin, Darexaban, Nafamostat, Gabexate, Sivelestat, Melagatran, Cholesterol sulfate, Dabigatran, Fondaparinux, Desirudin, Betrixaban, CGS-27023, GW-813893, Berotralstat, Evolocumab, Conestat alfa, Rosmarinic acid, Alpha-1 antitrypsin, Alpha-2 antiplasmin, BIA 10-2472, C1-inhibitor, Camostat, Cospin, CU-2010, CU-2020, Kallistatin, Kazal domain, Maspin, Methoxy arachidonyl fluorophosphonte, Microviridin, Plasminogen activator inhibitor-1, Plasminogen activator inhibitor-2, PMSF, Protein C inhibitor, Protein Z-related protease inhibitor, SERPINA9, SERPINB1, SERPINB3, SERPINB4, SERPINB6, SERPINB7, SERPINB8, SERPINB9, SERPINB13, SERPINE2, SPINT1, Spaostat, and Uterine Serpin.

In some embodiments, the serine protease inhibitor of the methods of the disclosure includes nafamostat mesylate (Futhan), camostat mesilate (FOY 305), gabexate mesilate (FOY) or derivatives, serine protease inhibitor Kazal-type 1 (SPINK1), tranexamic acids, granzyme A, granzyme B, UAMC-00050, 4-(2-minoethyl) benzenesulfonyl fluoride hydrochloride (AEBSF), soy bean trypsin inhibitor, meprin inhibitors, setmelanotide, or alpha-1-antitrypsin. In some embodiments, the serine protease inhibitor can include a derivative of any one of the serine protease inhibitors described herein.

### Pharmaceutical Compositions for the Treatment of Age-Related Conditions

The methods described herein include the use of pharmaceutical compositions comprising one or more of serine protease inhibitors as an active ingredient.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Pharmaceutical compositions are typically formulated to be compatible with its intended route of administration. Examples of routes of administration include parenteral, e.g., intravenous, subcutaneous, oral (e.g., capsules or inhalation), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a pharmaceutical composition as described herein can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

The pharmaceutical compositions can also be prepared in the form of suppositories (e.g., with conventional suppository bases such as cocoa butter and other glycerides) or retention enemas for rectal delivery.

In one embodiment, the pharmaceutical compositions are prepared with carriers that will protect the pharmaceutical compositions against rapid elimination from the body, such as a controlled release formulation, including implants and microencapsulated delivery systems. In some embodiments, the pharmaceutical compositions include a serine protease inhibitor that is linked, conjugated, or fused to another molecule. In some embodiments, the other molecule changes a property of the pharmaceutical composition. In some embodiments, pharmaceutical compositions can be delivered by using nanoparticle encapsulation. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Methods of Decreasing Serine Protease Activity Outside of the Gastrointestinal (GI) Tract

Aging and/or age-related diseases or conditions can cause an increase in the permeability of the intestinal barrier to digestive serine proteases such that serine protease activity may be detectable in the circulation of the subject. Digestive enzymes can leak across the mucin-epithelial barrier into tissues and organs outside the pancreas and intestines where they may damage the extracellular matrix and cell membranes. In some embodiments, damage may include ectodomain receptor cleavage.

The digestive enzymes can cause multiple forms of tissue damage, including cleavage of membrane receptors (e.g. the insulin receptor, growth hormone receptor) and degradation of collagen in organs of the subject. Pancreatic trypsin can also activate prohormones and interfere with physiological signaling due to its ability to cleave a broad spectrum of humoral mediators as well as their receptors. Treatment with administration of a digestive enzyme inhibitor (e.g., serine protease inhibitor, e.g., trypsin inhibitor) can attenuate breakdown of the mucin barrier, reduce the accumulation of digestive enzymes in peripheral organs, as well as cleavage of collagen. For example, interventions against pancreatic trypsin outside the small intestine not only block activation of secondary proteases, but also maintain a spectrum of cell functions (including, but not limited to, immune responses, mitochondrial functions, stem cell proliferation and differentiation, DNA repair mechanisms, epigenetics, protein folding, intra-and inter-cellular signaling, and nutrient utilization).

The compositions described herein can be administered to a subject to treat or prevent diseases, disorder, or conditions described herein. In some embodiments, the present disclosure describes methods of reversing accumulation of a serine protease in an organ of a subject, reversing cellular damage in an organ of a subject, and/or preserving extracellular matrix in an organ of a subject, by selecting a subject at risk of damage to the organ, and administering a therapeutically effective amount of a serine protease inhibitor. In some embodiments, the present disclosure describes methods of treating a disease or condition of the brain, spinal cord, heart, muscle, kidney, liver, or lung.

In some embodiments, the present disclosure describes methods of decreasing serine protease activity outside a gastrointestinal (GI) tract of a subject. In some embodiments, the methods can inhibit or reduce activity of a serine protease outside a gastrointestinal (GI) tract of a subject, or reduce symptoms of biological aging in a subject. In some embodiments, the methods include administering to a subject in need thereof a therapeutically effective amount of a serine protease inhibitor that results in the decrease in the activity of the serine protease outside the GI tract.

In some embodiments, the methods include prophylactically treating age-related diseases or conditions. As used herein, the term “prophylactically treating” can refer to a taking preventative measures to preserve health or prevent the progression of or occurrence of a disease or condition (e.g., reversing accumulation of a serine protease in an organ of a subject, reversing cellular damage in an organ of a subject, and/or preserving extracellular matrix in an organ of a subject). For example, a subject can be prophylactically treated when the subject is at risk of experiencing a disease or condition (e.g., having biomarkers that increase susceptibility of a particular condition, e.g., dementia).

### Subject

As used herein, the term “subject” refers an organism, typically a mammal (e.g., a human). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

In some embodiments, the subject can be an animal, human or non-human. Non-limiting examples of non-human subjects can include mice, rats, hamsters, rabbits, cats, dogs, horses, pigs, donkeys, monkeys, and/or other non-human primates such as apes and lemurs.

In some embodiments, the subject is a human. In some embodiments, a human patient can be an adult human or juvenile human (e.g., human below the age of 18 years old). In some embodiments, the subject is a patient suffering from an aging-related disease, disorder, or condition. In some embodiments, the subject is a patient susceptible to an aging-related disease, disorder, or condition. In some embodiments, the subject is a patient displaying one or more signs or symptoms or characteristics of an aging-related disease, disorder, or condition. In some embodiments, the subject is displaying symptoms of an age-related disease, disorder, or condition when the subject is considered biologically aged, e.g., over 50 years old, over 55 years old, over 60 years old, over 65 years old, over 70 years old, over 75 years old, over 80 years old, over 85 years old, over 90 years old, or over 95 years old. In some embodiments, the subject is displaying symptoms of an age-related disease, disorder, or condition at time when the subject is not considered biologically aged, e.g., under 45 years old, under 40 years old, under 35 years old, under 30 years old, or under 25 years old. In some embodiments, the subject is an adult human over the age of 18 years old. In some embodiments, the subject is older than 20 years old. In some embodiments, the subject is older than 30 years old. In some embodiments, the subject is older than 40 years old. In some embodiments, the subject is older than 50 years old. In some embodiments, the subject is older than 60 years old. In some embodiments, the subject is older than 70 years old. In some embodiments, the subject is older than 80 years old. In some embodiments, the subject is older than 90 years old. In some embodiments, the subject is older than 100 years old.

### Treating Treatment

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse aging-supportive process occurring in said individual, or will enhance or increase an aging-suppressive process in said individual. A “therapeutically effective amount” of a composition described herein can reverse (in a therapeutic treatment) the development accumulation of a serine protease in an organ of a subject, reverse cellular damage in an organ of a subject, or preserve extracellular matrix structure in an organ of a subject. A therapeutically effective amount can include preserving the molecular structure of organ tissue as detected by hybridizing peptides that can bind to collagen structure at cleavage sites. A therapeutically effective amount administered to an individual to treat a disease or condition in that individual may be the same or different from a therapeutically effective amount administered for prophylactic purposes. The therapeutic methods described herein are not to be interpreted as, restricted to, or otherwise limited to a “cure” for aging; rather the methods of treatment are directed to the use of the described compositions to “treat” age-related conditions, i.e., to effect a desirable or beneficial change in the health of an individual who has an age-related condition, such as but not limited to accumulation of a serine protease in an organ of a subject, reverse ongoing extracellular matrix protein (e.g., collagen) cleavage, cellular damage (e.g., membrane receptor cleavage) and cellular dysfunction (e.g., reduced integrin attachment to the extracellular matrix and intracellular integrin signaling) in an organ of a subject, or preserve extracellular matrix in an organ of a subject. As is understood in the art, an effective amount of a serine protease inhibitor may vary, depending on, inter alia, patient history as well as other factors such as the type (and/or dosage) of serine protease inhibitor used.

The phrases “reduced”, “decreased”, “a reduced level”, or “a decreased level” and similar phrases generally refer to a reduction or decrease of at least 1% (e.g., at least 2%, at least 4%, at least 6%, at least 8%, at least 10%, at least 12%, at least 14%, at least 16%, at least 18%, at least 20%, at least 22%, at least 24%, at least 26%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference level or value. The phrases “increased”, “greater”, “an increased level”, or “a greater level” and similar phrases generally refer to an increase of at least 1% (e.g., at least 2%, at least 4%, at least 6%, at least 8%, at least 10%, at least 12%, at least 14%, at least 16%, at least 18%, at least 20%, at least 22%, at least 24%, at least 26%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, 100%, 150%, 200%, or more) as compared to a reference level or value.

In some embodiments, a therapeutically effective amount may be formulated and/or administered in a plurality of doses, for example, as part of a dosing regimen. In some embodiments, effective amounts and schedules for administering the serine protease inhibitor described herein may be determined empirically, and making such determinations is within the skill in the art. Those skilled in the art will understand that the dosage that must be administered will vary depending on, for example, the subject that will receive the serine protease inhibitor disclosed herein, the route of administration, the particular type of serine protease inhibitor, and other drugs being administered to the subject. In some embodiments, the administration of a therapeutically effective amount comprises chronic administration, whereas in other embodiments the administration of a therapeutically effective amount comprises a scheduled administration.

In some embodiments, the scheduled administration includes a predetermined schedule. Non-limiting examples of a scheduled basis include every other day, every two days, every three days, every four days, every five days, every six days, or once a week. Other non-limiting examples of a scheduled basis include one day on: six days off, two days on: five days off, three days on: four days off, four days on: three days off, five days on: two days off, six days on: one day off. Yet further non-limiting examples of a scheduled basis include two days on: one day off, two days on: two days off, two days on: three days off, two days on—four days off, two days on: five days off, three days on: one day off, three day's on: two days off, three days on: three days off, three days on: four days off, four days on: one day off, four days on—two days off, four days on: three days off, five days on: one day off, five days on: two days off, six days on: one day off. Yet further non-limiting examples of a scheduled basis include one day out of every seven days, two days out of every seven days, three days out of every seven days, four days out of every seven days, five days out of every seven days, or six out of every seven days. The method may comprise administering the composition by a weekly protocol consisting of daily administration of a maintenance dose composition for 3-5 consecutive days followed by no administration for 1-3 consecutive days.

In some embodiments, the subject can be administered the serine protease inhibitor over an extended period of time (e.g., over a period of at least 1 week, 2 weeks, 3 weeks, 1 month, 2 months, 3 months, 4 months, 5 months, 6 months, 7 months, 8 months, 9 months, months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of aging). As described herein, a skilled medical professional can also change the identity and number (e.g., increase or decrease) of the serine protease inhibitor administered to the subject and can also adjust (e.g., increase or decrease) the dosage or frequency of administration of the serine protease inhibitor to the subject based on an assessment of the effectiveness of the treatment. In some embodiments, the serine protease inhibitor is administered for more than 1 week. In some embodiments, the serine protease inhibitor is administered for more than 2 weeks. In some embodiments, the serine protease inhibitor is administered for more than 4 weeks. In some embodiments, the serine protease inhibitor is administered for more than one month, more than two months, more than three months, more than four months, more than five months, more than six months, more than seven months, more than eight months, more than nine months, more than 10 months, more than 11 months, more than 12 months, or longer.

In some embodiments, the serine protease inhibitor can be administered at a concentration that is lower than the serine protease concentration within the GI tract. In some embodiments, the serine protease inhibitor can be administered at a concentration that is less than 10% of the serine protease concentration of the GI tract. In some embodiments, the serine protease inhibitor can be administered at a concentration that is less than 9%, 8%, 7%, 6%, 5%, 4%, 3%, 2%, or 1% of the serine protease concentration of the GI tract. For example, a small molecular weight serine protease competitive inhibitor (e.g., TXA or FOY), is administered at a concentration (e.g., 10 μM) below the serine protease concentration inside the small intestine (e.g., 100 μM), but which matches and/or exceeds the protease concentration in the plasma (e.g., 5 μM). In some embodiments, the serine protease inhibitor administered blocks the activity of serine proteases in the plasma. In some embodiments, the majority of the digestive activity of the small intestine is preserved. In some embodiments, the concentration of the serine protease inhibitor does not interfere or reduce digestion or functional activity of the stomach and/or small intestine. The serine protease concentration within the GI tract can be determined empirically or it may be determined by consultation to a standardized and accepted source of such information.

In some embodiments, the concentration of the serine protease inhibitor to be administered to a subject can be determined by measuring serine protease activity in the subject. In some embodiments, the concentration of the serine protease inhibitor to be administered to a subject can be determined by measuring serine protease activity in the subject at a specific time point. In some embodiments, the concentration of the serine protease inhibitor to be administered to a subject can be determined by measuring serine protease activity outside the GI tract (e.g., in the plasma, in the peripheral tissue) of the subject. In some embodiments, serine protease concentration within the GI tract is determined by mass spectrometry determination of peptide incidence in plasma. For example, a sample of a patient's plasma can be run through a mass spectrometer and proteolysis of the plasma proteins can be determined. In some embodiments, serine protease concentration can be determined by receptor cleavage with antibody against extracellular domains using cells harvested from the subject or the subject's plasma or other body fluid (e.g., lymph fluid).

In some embodiments, the serine protease inhibitor can be administered at a concentration that is lower than the serine protease concentration within the GI tract but higher than the serine protease concentration outside the GI tract (e.g., in plasma, or in peripheral tissues). In some embodiments, the serine protease inhibitor can be administered at a concentration that is about the same as the serine protease concentration outside the GI tract. In some embodiments, the serine protease inhibitor can be administered at a concentration that is higher than the serine protease inhibitor concentration outside the GI tract. In some embodiments, the serine protease inhibitor can be administered at a concentration that is at least 60%, at least 65%, at least 70%, at least 75%, at least 80%, at least 85%, at least 90%, at least 95%, or 100% the concentration of serine protease in the subject's plasma and/or tissue.

In some embodiments, the serine protease inhibitor can be administered at a concentration of less than 10 mM, less than 9 mM, less than 8 mM, less than 7 mM, less than 6 mM, less than 5 mM, less than 4 mM, less than 3 mM, less than 2 mM, less than 1 mM, less than 0.5 mM, or less than 0.1 mM.

In some embodiments, the serine protease inhibitor can be administered at a concentration of less than 50 μM (e.g., less than 48 μM, less than 46 μM, less than 44 μM, less than 42 μM, less than 40 μM, less than 38 μM, less than 36 μM, less than 34 μM, less than 32 μM, less than 30 μM, less than 28 μM, less than 26 μM, less than 24 μM, less than 22 μM, less than 20 μM, less than 18 μM, less than 16 μM, less than 14 μM, less than 12 μM, less than 10 μM, less than 8 μM, less than 6 μM, less than 5 μM, less than 4 μM, less than 3 μM, less than 2 μM, less than 1 μM, less than 0.5 μM, less than 0.25 μM, or less than 0.1 μM). In some embodiments, the serine protease inhibitor is administered at a concentration of less than 5 μM. In some embodiments, the serine protease inhibitor is administered at a concentration of less than 1 μM (e.g., less than 0.8 μM, less than 0.6 μM, less than 0.4 μM, less than 0.2 μM, less than 0.1 μM, less than 90 nM, less than 80 nM, less than 70 nM, less than 60 nM, less than 50 nM, less than 40 nM, less than 30 nM, less than 20 nM, less than 10 nM, less than 5 nM, less than 3 nM, less than 1 nM, less than 0.8 nM, less than 0.6 nM, less than 0.4 nM, less than 0.2 nM, less than 0.1 nM, less than 90 pM, less than 80 pM, less than 70 pM, less than 60 pM, less than 50 pM, less than 40 pM, less than 30 pM, less than 20 pM, less than 10 pM, less than 5 pM, less than 3 pM, less than 1 pM, less than 0.8 pM, less than 0.6 pM, less than 0.4 pM, less than 0.2 pM, or less than 0.1 pM).

In some embodiments, the serine protease inhibitor can be administered according to the patient's weight. In some embodiments the serine protease inhibitor can be administered anywhere between 0.01 and 1.0 gm/kg/day. In some embodiments, a therapeutically effective amount of serine protease inhibitor can include 0.01 gm/kg/day, 0.02 gm/kg/day, 0.03 gm/kg/day, 0.04 gm/kg/day, 0.05 gm/kg/day, 0.06 gm/kg/day 0.07 gm/kg/day, 0.08 gm/kg/day, 0.09 gm/kg/day, 0.1 gm/kg/day, 0.11 gm/kg/day, 0.12 gm/kg/day, 0.13 gm/kg/day, 0.14 gm/kg/day, 0.15 gm/kg/day, 0.16 gm/kg/day, 0.17 gm/kg/day, 0.18 gm/kg/day, 0.19 gm/kg/day, 0.20 gm/kg/day, 0.21 gm/kg/day, 0.22 gm/kg/day, 0.23 gm/kg/day, 0.24 gm/kg/day, 0.25 gm/kg/day, 0.26 gm/kg/day, 0.27 gm/kg/day, 0.28 gm/kg/day, 0.29 gm/kg/day, 0.30 gm/kg/day, 0.31 gm/kg/day, 0.32 gm/kg/day, 0.33 gm/kg/day, 0.34 gm/kg/day, 0.35 gm/kg/day, 0.36 gm/kg/day, 0.37 gm/kg/day, 0.38 gm/kg/day, 0.39 gm/kg/day, 0.40 gm/kg/day, 0.41 gm/kg/day, 0.42 gm/kg/day, 0.43 gm/kg/day, 0.44 gm/kg/day, 0.45 gm/kg/day, 0.46 gm/kg/day, 0.47 gm/kg/day, 0.48 gm/kg/day, 0.49 gm/kg/day, 0.50 gm/kg/day, 0.51 gm/kg/day, 0.52 gm/kg/day, 0.53 gm/kg/day, 0.54 gm/kg/day, 0.55 gm/kg/day, 0.56 gm/kg/day, 0.57 gm/kg/day, 0.58 gm/kg/day, 0.59 gm/kg/day, 0.60 gm/kg/day, 0.61 gm/kg/day, 0.62 gm/kg/day, 0.63 gm/kg/day, 0.64 gm/kg/day, 0.65 gm/kg/day, 0.66 gm/kg/day, 0.67 gm/kg/day, 0.68 gm/kg/day, 0.69 gm/kg/day, 0.70 gm/kg/day, 0.71 gm/kg/day, 0.72 gm/kg/day, 0.73 gm/kg/day, 0.74 gm/kg/day, 0.75 gm/kg/day, 0.76 gm/kg/day, 0.77 gm/kg/day, 0.78 gm/kg/day, 0.79 gm/kg/day, 0.80 gm/kg/day, 0.81 gm/kg/day, 0.82 gm/kg/day, 0.83 gm/kg/day, 0.84 gm/kg/day, 0.85 gm/kg/day, 0.86 gm/kg/day, 0.87 gm/kg/day, 0.88 gm/kg/day, 0.89 gm/kg/day, 0.90 gm/kg/day, 0.91 gm/kg/day, 0.92 gm/kg/day, 0.93 gm/kg/day, 0.94 gm/kg/day, 0.95 gm/kg/day, 0.96 gm/kg/day, 0.97 gm/kg/day, 0.98 gm/kg/day, 0.99 gm/kg/day, 1.0 gm/kg/day.

In some embodiments, depending on the properties of the serine protease inhibitor, a therapeutically effective amount of serine protease inhibitor can include amounts higher than 1.0 gm/kg/day. For example, a therapeutically effective amount of serine protease inhibitor can include, 1 gm/kg/day, 2 gm/kg/day, 3 gm/kg/day, 4 gm/kg/day, 5 gm/kg/day, 6 gm/kg/day, 7 gm/kg/day, 8 gm/kg/day, 9 gm/kg/day, 10 gm/kg/day, 11 gm/kg/day, 12 gm/kg/day, 13 gm/kg/day, 14 gm/kg/day, 15 gm/kg/day, 16 gm/kg/day, 17 gm/kg/day, 18 gm/kg/day, 19 gm/kg/day, 20 gm/kg/day, 21 gm/kg/day, 22 gm/kg/day, 23 gm/kg/day, 24 gm/kg/day, 25 gm/kg/day, 26 gm/kg/day, 27 gm/kg/day, 28 gm/kg/day, 29 gm/kg/day, 30 gm/kg/day, 31 gm/kg/day, 32 gm/kg/day, 33 gm/kg/day, 34 gm/kg/day, 35 gm/kg/day, 36 gm/kg/day, 37 gm/kg/day, 38 gm/kg/day, 39 gm/kg/day, 40 gm/kg/day, 41 gm/kg/day, 42 gm/kg/day, 43 gm/kg/day, 44 gm/kg/day, 45 gm/kg/day, 46 gm/kg/day, 47 gm/kg/day, 48 gm/kg/day, 49 gm/kg/day, 50 gm/kg/day, 51 gm/kg/day, 52 gm/kg/day, 53 gm/kg/day, 54 gm/kg/day, 55 gm/kg/day, 56 gm/kg/day, 57 gm/kg/day, 58 gm/kg/day, 59 gm/kg/day, 60 gm/kg/day, 61 gm/kg/day, 62 gm/kg/day, 63 gm/kg/day, 64 gm/kg/day, 65 gm/kg/day, 66 gm/kg/day, 67 gm/kg/day, 68 gm/kg/day, 69 gm/kg/day, 70 gm/kg/day, 71 gm/kg/day, 72 gm/kg/day, 73 gm/kg/day, 74 gm/kg/day, 75 gm/kg/day, 76 gm/kg/day, 77 gm/kg/day, 78 gm/kg/day, 79 gm/kg/day, 80 gm/kg/day, 81 gm/kg/day, 82 gm/kg/day, 83 gm/kg/day, 84 gm/kg/day, 85 gm/kg/day, 86 gm/kg/day, 87 gm/kg/day, 88 gm/kg/day, 89 gm/kg/day, 90 gm/kg/day, 91 gm/kg/day, 92 gm/kg/day, 93 gm/kg/day, 94 gm/kg/day, 95 gm/kg/day, 96 gm/kg/day, 97 gm/kg/day, 98 gm/kg/day, 99 gm/kg/day, or 100 gm/kg/day.

### Administration

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, enteral, parenteral, etc. In some particular embodiments, administration may be bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, topical, tracheal (e.g., by intratracheal instillation), vaginal, vitreal, by patch, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may involve methods of delivery that include, but are not limited to, use of external and/or implanted infusion pumps, liquid formulation, capsulated formulation, or slow release encapsulation.

In some embodiments, the serine protease inhibitor administration can be ocular, oral, parenteral, bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, tracheal (e.g., by intratracheal instillation), vaginal, or vitreal. In some embodiments, the serine protease inhibitor is administered by enteral administration, intraperitoneal administration, intravenous administration, intramuscular administration, subcutaneous administration, intracutaneous administration, oral administration, intranasal administration, intrapulmonary administration, intrarectal administration, or a telemetry controlled external or implanted infusion pump. In some embodiments, the serine protease inhibitor is administered by oral administration. In some embodiments, the serine protease inhibitor is administered by a telemetry controlled infusion pump. In some embodiments, the telemetry controlled infusion pump is directed toward a target tissue. In some embodiments, the target tissue can include adipose tissue, pancreatic tissue, liver tissue, kidney tissue, lung tissue, vasculature, bone tissue, central nervous system (CNS) tissue, eye tissue, muscle tissue, and secondary lympho-organ tissue. In some embodiments, the target tissue can include veins, arteries, lymphatics, cerebral spinal fluid, subcutaneous tissue, or joints. In some embodiments, the telemetry controlled infusion pump is directed toward a target organ. In some embodiments, the target organ can include the brain, spinal cord, heart, kidney, muscle, liver, lung, pancreas, or any other organ. In some embodiments, the serine protease inhibitor is administered in a form of eye drops or liposome composition.

In some embodiments, the subject can be administered more than one serine protease inhibitor. In some embodiments, the administration of the one or more serine protease inhibitors is sequential administration (e.g., one serine protease inhibitor is administered, stopped, and a second, different serine protease inhibitor is administered). In some embodiments, the subject can be administered a combination of serine protease inhibitors at the same time.

In some embodiments, the methods of the disclosure can be administered before, in conjunction with, or after other methods or therapeutic treatments, either for the condition being treated by administration of the serine protease inhibitor, or another condition. In a non-limiting example, a subject can be treated with surgery for a cancerous mass in the intestine, and after the surgery the subject can be administered a serine protease inhibitor to limit leakage of proteases from the intestine. Preventative/prophylactic administration of serine protease inhibitors can be used to slow and/or prevent autodigestion of the subject's organs due to the intestinal permeability. In another non-limiting example, the subject may be administered a serine protease inhibitor for treatment of suspected dementia, while the subject is concurrently taking another medication either for the subject's dementia or for another condition (e.g., diabetes).

In some embodiments, serine protease activity is increased in a postprandial period. To block this activity, the serine protease inhibitor can be administered before food intake (e.g., eating). In diabetics or pre-diabetics, this postprandial period of elevated serine protease activity is longer than in non-diabetics, and may last for several hours. In some embodiments, the amount or concentration of serine protease inhibitor administration will depend on measurements of protease activity in plasma or in peripheral tissues, like abdominal fluid, heart, brain, intestine, kidney, liver, lung, eye, or other tissues disclosed herein. In some embodiments, the serine protease inhibitor is administered during a diurnal cycle, wherein the serine protease inhibitor administration depends on the measured serine protease activity in the subject.

### Organs/Conditions/Diseases

In some embodiments, the method provided herein can reduce symptoms of biological aging in a subject. In some embodiments, the subject can display one or more signs or symptoms or characteristics of an aging-related disease, disorder, or condition.

In some embodiments, the methods involved selecting a subject at risk of damage to an organ. The subject can be at risk of damage to the organ because the subject is exhibiting symptoms consistent with a known disease or condition that affects that organ. The subject can be at risk of damage to the organ because the subject has a biomarker known to predispose the subject to a known disease or condition that affects that organ. The subject can be at risk of damage to the organ because the subject has a family history that would predispose them to a known disease or condition that affects that organ. In some embodiments, the subject demonstrates symptoms and/or biomarkers of elevated serine protease activity outside the GI tract (e.g., in plasma, or in peripheral tissue).

For the methods disclosed herein, the organ may be selected from the group consisting of the brain, spinal cord, heart, kidney, muscle, intestine, liver, eye, skeletal muscle, abdominal and subcutaneous adipose tissue, small and large intestinal wall, connective tissue (including mesentery), breast tissue, tissues from the male and female reproductive organs, bone, cartilage, ear, nasal tract, and lung. In some embodiments, the organ may be selected from the group consisting of brain, heart, intestine, and muscle. In some embodiments, the organ is the heart. In some embodiments the organ is the brain. In some embodiments, the organ is the kidney. In some embodiments, the organ is the liver. In some embodiments, the organ is the intestine (e.g., small intestine and/or large intestine). In some embodiments, the organ is the eye. In some embodiments, the organ is the lung. For clarity, the methods of the disclosure are not intended to treat shock (e.g., septic shock) or HIV. Further, the methods of the disclosure may further include a step of screening a subject for shock (e.g., septic shock) and/or HIV, and not administering a serine protease inhibitor to the subject if the subject is currently experiencing shock or biomarkers of HIV.

In some embodiments, the aging-related disease can include Alzheimer's disease, age-related loss of neuronal function including but not limited to memory loss, loss of balance, and sensory function, pain, including but not limited to lower back pain, aneurysm, chronic venous disease, cystic fibrosis, fibrosis in pancreatitis, glaucoma, hypertension, idiopathic pulmonary fibrosis, inflammatory bowel disease, intervertebral disc degeneration, osteoarthritis, type 2 diabetes mellitus, adipose atrophy, lipodystrophy, atherosclerosis, cataracts, COPD, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, age-related macular degeneration, cachexia, glomerulosclerosis, liver cirrhosis, nonalcoholic fatty liver disease (NAFLD), nonalcoholic steatohepatitis (NASH), hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, osteoporosis, Huntington's disease, spinocerebellar ataxia, mental disorders, neurodegeneration, epilepsy or other seizure disorders, stroke, cancer, dementia, including frontomemporal dementia, meningitis, encephalitis, vascular disease, coronary heart disease, angina, unstable angina, heart failure, valve disease, high blood pressure, heart arrhythmias, endocardidtis, pericardial disease, cardiomyopathy, infection susceptibility, chronic inflammation, renal dysfunction, rheumatoid arthritis, inflammatory bowel disease, lupus erythematosus, lupus nephritis, diabetic nephropathy, CNS injury, amyotrophic lateral sclerosis, fibromyalgia, myositis, including polymyositis and dermatomyositis, muscular dystrophy, myasthenia gravis, rhabdomyolysis, sarcopenia, Charcot-Marie-Tooth disease, myopathy, peripheral neuropathy, and spinal muscular atrophy, acute kidney injury, kidney stones, kidney infections, including pyelonephritis, kidney cysts, kidney cancer, conditions where the subject is in need of renal dialysis, pancreatic cancer, Crohn's disease, multiple sclerosis, Guillain-Barre syndrome, psoriasis, Grave's disease, ulcerative colitis, mood disorders and/or cognitive impairment.

### Kits

Also provided herein are kits for the use in the methods described herein. For example, the kids can include a composition comprising a serine protease inhibitor for oral administration. Instructions for use can also be included in the kits.

### EXAMPLES

### Example 1: Sample Collection and Serine Protease Inhibition in Various Organs in Rats

### Animals and Tissue Collection

Male Wistar rats (Harlan Sprague Dawley Inc., Indianapolis, IN) at maturity (4 months, 300 to 350 gm) and old age (24 months, 375 to 450 gm) were included in the study. The animals were maintained on standard laboratory chow (8604 Teklad rodent diet; Harlan Laboratories, Indianapolis, IN) without restriction and water ad libitum and maintained in separated room without pathogen-free conditions. They were confirmed to exhibit normal mobility, water and food consumption and fecal material discharge. Animals that exhibited signs of morbidities were excluded. A subgroup of old animals was given a serine protease inhibitor (tranexamic acid, 14 days) in drinking water (137 mM, exchanged daily) which at a minimum fluid consumption of 40 ml/day amounts to a minimum dose of 0.39 gm/kg/day for 350 gm body weight (BW).

A femoral venous catheter was placed after general anesthesia (pentobarbital sodium, 50 mg/kg [Abbott Laboratories, North Chicago, IL], intramuscularly after local anesthesia with 2% lidocaine HCl [Hospira, Inc, Lake Forrest, IL]). Tissues (intestine, liver, lung, heart, kidney, brain, mesentery (n=1)) were immediately collected after euthanasia (Beuthanasia i.v., 120 mg/kg, Schering-Plough Animal Health Corp, Union, NJ), fixed (formalin, 10%, neutral buffered, 1 hr), postfixed (in fresh formalin solution, 24 hrs), and stored in formalin (10%). The period between initial anesthesia and fixation of the mesentery was kept below 60 minutes to minimize activation or de novo syntheses of MMPs during the tissue collection.

### Tissue Sections

Formalin fixed tissues were cut into 40 μm sections with a vibratome (Pelco Lancer Vibratome Series 1000).

In addition, to generate thinner sections for the intestine, a segment of the upper jejunum was embedded in Araldite resin (Polysciences, Washington, PA) and cut again into 1 μm section (Ultramicrotome, LKB Ultratome Nova, with diamond knife). The resin was removed (Maxwell Solution (1 dip)), rinsed in tap water, incubated in hydrogen peroxide (4%, for 1 minute), rinsed (in phosphate buffer), and immunolabeled for trypsin.

### Serine Protease Immunohistochemistry

To determine on the tissue sections the immunolabel density and distribution of serine proteases, pancreatic trypsin MoAb (D-1): sc-137077 (Santa Cruz) primary antibody was used, followed by secondary antibodies (MP-7601 for anti-rabbit IgG; MP-7602 for anti-mouse IgG; ImmPRESS Excel staining kit peroxidase). Two substrate colors were used, red (ImmPact™ AEC Substrate kit peroxidase, sk-4205; Vector® Laboratories) and brown (ImmPACT™ DAB Substrate kit peroxidase, sk4105; and Vectorstain Elite ABC-HRP Kit, Vector® Laboratories). Sections without primary antibody served as controls. No counterstain was applied to facilitate quantitative label intensity measurements and since cellular and vascular structures are readily identified. The concentrations and exposure of primary and secondary antibodies applied to the sections were adjusted (24 hrs and according to protocol by Vector® Laboratories, respectively) to achieve full penetration of the antibodies into the tissue sections. All procedures were carried out under standardized conditions to permit quantitative comparison of label densities.

### Whole Mount Tissue Labeling

Small intestine: Full thickness tissue blocks of the wall of proximal jejunum (3 by 5 mm) were fixed from all sides in 10% formaldehyde. Serine proteases were detected with primary and secondary antibodies labeled with DAB (Peroxidase Substrate Kit, ab64238, ABCAM).

The mucin-containing mucus layer on the epithelial cells of the small intestine was stained using alcian blue (pH 2.5, kt 003; Diagnostic BioSystems, Pleasanton, CA) followed by a rinse in distilled water and mounted on a microscope slide (Vector Mount AQ Aqueous Mounting Medium, Vector Laboratories, Burlington, CA).

Mesentery: The trypsin distribution in intact mesentery sectors was delineated by biotin/avidin immunolabeling with MoAb (D-1), secondary antibody (anti-mouse IgG, MP-7602, ImmPRESS Excel staining kit peroxidase, Vector® Laboratories) with brown substrate (ImmPACT DAB sk-4105).

### Collagen Fragment Labeling

After removal of resin, 1 um sections were labeled with biotin conjugated collagen hydridizing peptides (B-CHP) that bind unfolded collagen by triple helix formation and localize molecular level failure of collagen with high specificity.

Tissue sections were stained with B-CHP (stock solution, 150 mM; final applied solution 7.5. mM). The trimeric CHP are thermally dissociated to monomers before use (80° C. for 10 min), the hot CHP solution is quickly cooled to room temperature (by immersion into 4° C. water for 15 sec) and diluted and immediate applied to the section (dead time <1 min). In this way, most CHP peptides were expected to remain as active monomers during the staining process, based on kinetic studies on CHP triple helix folding. Sections were incubated overnight at room temperature, unbound B-CHP was removed by washing (3 times in 1 ml of 1×PBS for 30 min at room temperature). To visualize the B-CHP, the tissue sections were incubated with streptavidin peroxidase (sk-5704, Vector® Laboratories, according to manufacturer instructions) and then to a substrate (ImmPact AEC Substrate Kit Peroxidase; sk-4205, Vector Laboratories) at room temperature (for periods between 1 and 10 min depending on the tissue). The B-CHP label intensity on the sections was recorded by digital microscopy.

To label thin (1 μm) sections of the small intestine for both mucin and trypsin, the fixed intestine was immersed in the primary antibody against trypsin and stained with DAB substrate. Thereafter the tissue was embedded in resin and sectioned. The mucin label (alcian blue), was applied to the thin section, cover slipped and imaged.

### Glucose Analysis

At the time of tissue collection, fresh femoral arterial blood was used to measure the blood glucose level (Contour, Bayer Diabetes Care, Tarrytown, NY) and the percent of glycated hemoglobin levels (AIC Home Test; Bristol-Myers-Squibb Co; NY, NY).

Insulin Receptor Density (in mesentery)

Measurement of insulin receptor cleavage was carried out by labeling its ectodomain with an antibody. Fixed tissue sections (10% formalin, neutral buffered) were identified with a primary antibody against the extracellular domain of the insulin receptor (Rα, N-20, sc-710 polyclonal antibody mapping to the N-terminus, Santa Cruz Biotech). A biotin/avidin technique with peroxidase enzyme substrate (ImmPACT AEC substrate kit sk-4205;Vector®Laboratories.) was used to visualize the primary antibody. Sections without primary antibody were used as negative controls.

### Digital Image Analysis

Images of the immunolabel density were recorded at different magnifications, from relatively low power overviews of the tissue (10× objective, numerical aperture 0.25) to higher magnification of single cells (at 60× oil immersion objective, numerical aperture 1.4). The images were recorded under standard light conditions under fixed settings of the substage condenser with a digital camera (Spot Insight GIGABIT camera, Sterling Height), so that the camera serves as a quantitative light intensity meter. Images were analyzed on a laboratory computer to minimize operator error (NIH Image, 1.61, public domain software, spatial resolution of 640×480 pixel).

The density of the immune substrate label was measured in form of light absorption (A), such that A=ln(I/Io). I is the light intensity over the tissue and Io is the incident light intensity without tissue. For each organ, the mean label density per group (with 3 animals per group) was determined from the average label density per animal (determined from 5 organ tissue section/animal, 30 images/section).

### Statistics

Measurements were summarized as mean±standard deviation. For comparisons between two groups an unpaired two-tailed Student's t-test was used. Analysis of variance (ANOVA) was used to test for differences in outcomes of interest among groups. Results were determined to be significant at p<0.05. Bonferroni's post-hoc multiple comparison test was used to determine significance between individual groups. The number of animals used was estimated to minimize the number of experimental animals required to obtain a statistically conclusive result, assuming equal variances among groups, α=0.05 and β=1-0.9. Animals were randomly assigned to study or control groups. No animals were excluded from analysis, and studies were blinded.

### Results—Heart

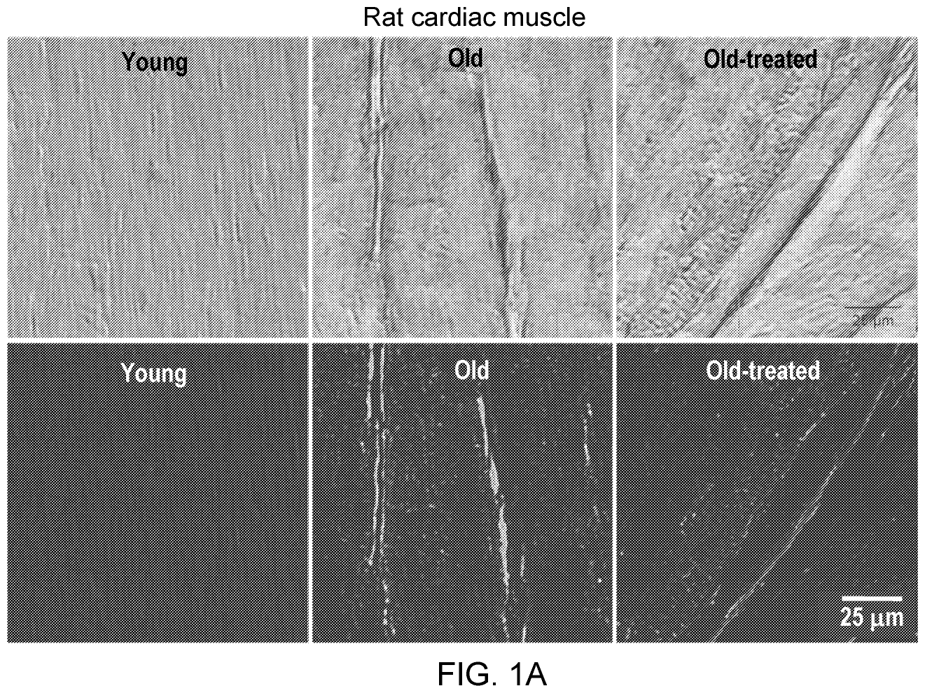

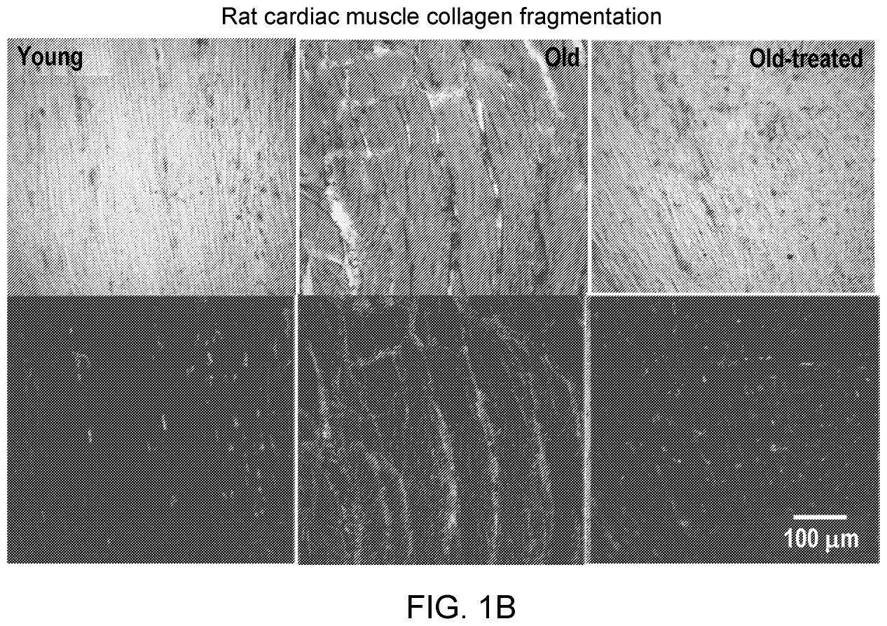

Immunohistochemistry was performed with a mAB against pancreatic trypsin in rat cardiac muscle under standard conditions for young, old, and old-treated groups. Two-week treatment with an oral trypsin inhibitor reduces pancreatic trypsin accumulation on old rats (100 weeks; “old-treated”) to similar levels seen in young rats (16 weeks) (FIG. 1A). Further, two-week treatment with an oral trypsin inhibitor reduces collagen fragmentation in cardiac muscle in old-treated rats below levels seen in old rats to levels similar to those seen in young rats (16 weeks) (FIG. 1B). Results show that the serine protease inhibitor intervention prevents collagen fragmentation in aged rats and improves organ function.

### Results—Brain

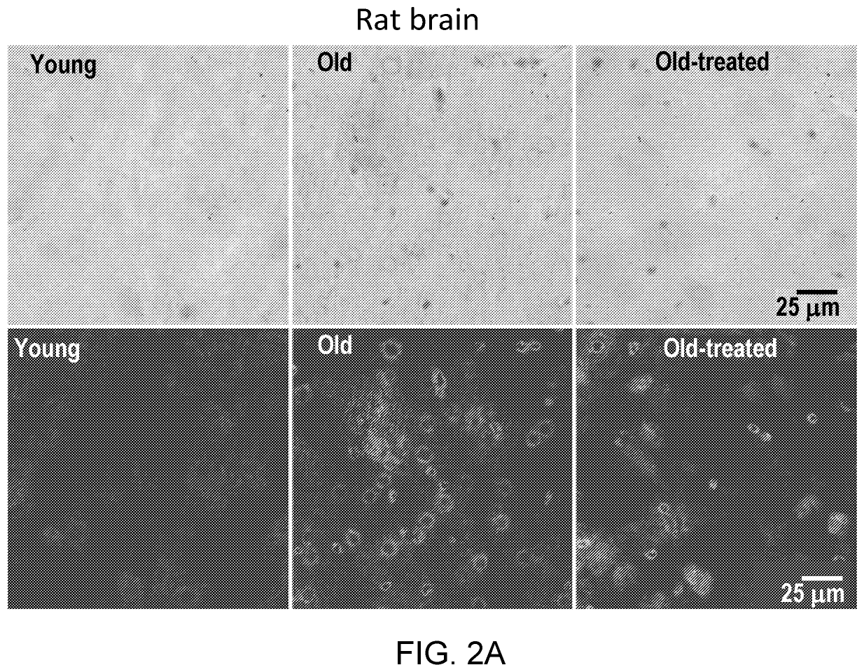

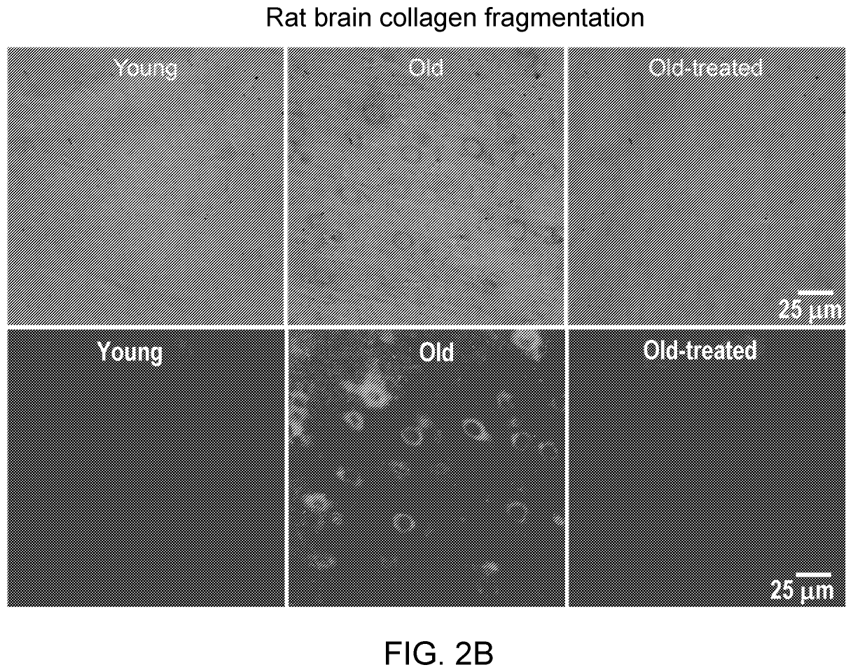

Immunohistochemistry was performed with a mAB against pancreatic trypsin in rat brain under standard conditions for young, old, and old-treated groups. Two-week treatment with an oral trypsin inhibitor reduces pancreatic trypsin accumulation on old rats (100 weeks; “old-treated”) to similar levels seen in young rats (16 weeks) (FIG. 2A). Further, two-week treatment with an oral trypsin inhibitor reduces collagen fragmentation in rat brain in old-treated rats below levels seen in old rats to levels similar to those seen in young rats (16 weeks) (FIG. 2B). Results show that the serine protease inhibitor intervention prevents collagen fragmentation in aged rats and improves organ function.

### Results—Intestine

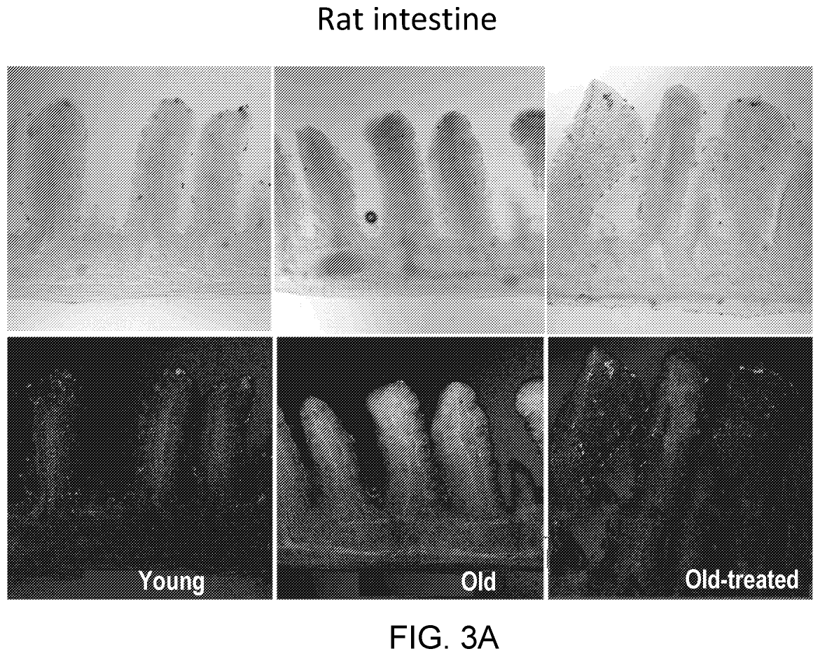

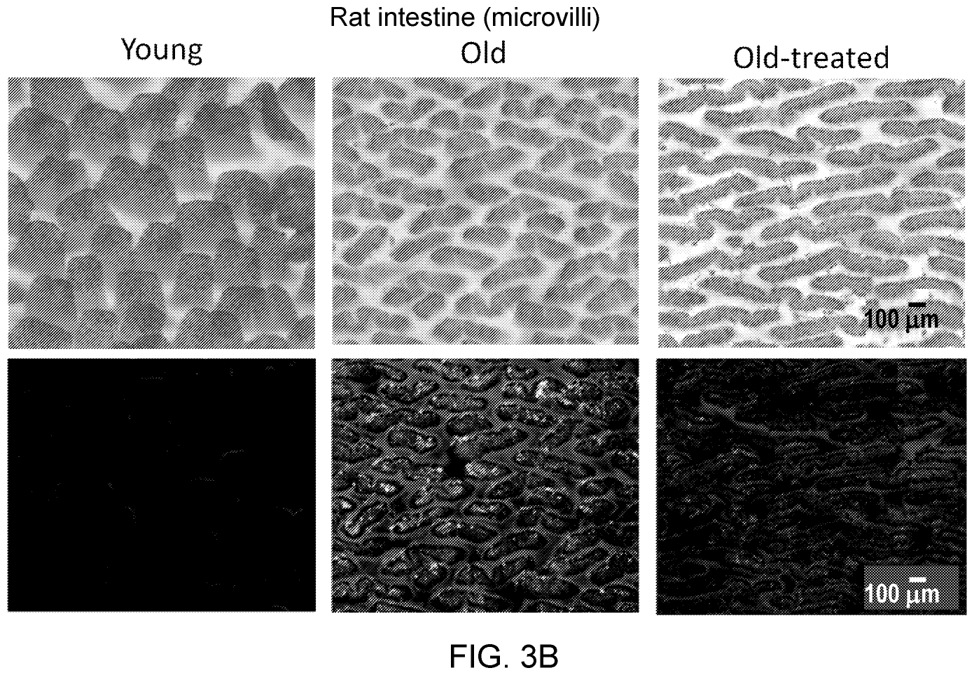

Immunohistochemistry was performed with a mAb against pancreatic trypsin in rat intestine under standard conditions for young, old, and old-treated animal groups. Two-week treatment with an oral trypsin inhibitor reduces pancreatic trypsin accumulation in old rats (100 weeks) to similar levels seen in young rats (16 weeks) (FIG. 3A).

The villi in the rat small intestine consist of elongated fold-shaped villi. The folds are aligned parallel with the long axis of the intestine. Mucin in the villi of the small intestine is a barrier for digestive enzymes. Mucin label density is significantly reduced in old rats, especially at the tip of the villi. Mucin label density was partially restored by two-week orally administered trypsin-inhibitors in old, treated rats (FIG. 3B).

### Example 2: Exemplary Treatment of Potential Conditions in Patients—Cardiac

A patient presents at the doctor with shortness of breath and fatigue. The doctor assesses the patient and determines that the patient likely has symptoms of heart failure. The doctor prescribes chronic administration of a pharmaceutical composition disclosed herein for the treatment of the heart failure symptoms. The patient's heart failure symptoms stabilize and start to improve.

### Example 3: Exemplary Treatment of Potential Conditions in Patients—Brain

A patient presents at the doctor with forgetfulness and memory problems. The doctor assesses the patient and determines that the patient likely has mild cognitive impairment. The doctor prescribes chronic administration of a pharmaceutical composition disclosed herein for the treatment of the mild cognitive impairment. The patient's mild cognitive impairment symptoms stabilize.

### Example 4: Exemplary Treatment of Potential Conditions in Patients—Kidney

A patient presents at the doctor with decreased urine output and fatigue. The doctor assesses the patient and determines that the patient likely has symptoms of kidney failure. The doctor prescribes chronic administration of a pharmaceutical composition disclosed herein for the treatment of the kidney failure symptoms. The patient's kidney failure symptoms improve.

### Example 5: Exemplary Treatment of Potential Conditions in Patients—Liver

A patient presents at the doctor with jaundice and nausea. The doctor assesses the patient and determines that the patient likely has symptoms of liver failure. The doctor prescribes chronic administration of a pharmaceutical composition disclosed herein for the treatment of the liver failure symptoms. The patient's liver failure symptoms stabilize.

### Example 6: Exemplary Treatment of Potential Conditions in Patients—Lung

A patient presents at the doctor with shortness of breath and cough. The doctor assesses the patient and determines that the patient likely has symptoms of lung disease. The doctor prescribes chronic administration of a pharmaceutical composition disclosed herein for the treatment of the lung disease symptoms. The patient's lung disease symptoms stabilize.

### Example 7: Exemplary Treatment of Potential Conditions in Patients—Intestine

A patient presents at the doctor with dyspepsia. bloating. and nausea. The doctor assesses the patient and determines that the patient likely has symptoms of intestinal perforation/leaky gut. The doctor prescribes chronic administration of a pharmaceutical composition disclosed herein for the treatment of the leaky gut symptoms. The patient's leaky gut symptoms stabilize.

### Example 8: Exemplary Treatment of Potential Conditions in Patients—Tremor

A patient presents at the doctor with shaking in the hands and difficulty writing or drawing. The doctor assesses the patient and determines that the patient likely has symptoms of tremor. The doctor prescribes chronic administration of a pharmaceutical composition disclosed herein for the treatment of the patient's tremor symptoms. One or more of the patient's tremor symptoms generally stabilize.

### Example 9: Exemplary Treatment of Potential Conditions in Patients—Unstable Blood Pressure

A patient presents at the doctor with unstable blood pressure. The doctor assesses the patient and determines that the patient has unstable blood pressure in need of treatment. The doctor prescribes chronic administration of a pharmaceutical composition disclosed herein for the treatment of the patient's unstable blood pressure. The patient's blood pressure stabilizes.

### Example 10: Exemplary Treatment of General Biological Aging in Older Patients

An older patient presents at the doctor with mild loss of muscle strength, mild loss of sight and hearing, and mild loss of energy compared to a few years ago. The doctor assesses the patient and determines that the patient does not currently meet the criteria for any particular disease or condition, and that these symptoms are likely due to biological aging. The doctor prescribes chronic administration of a pharmaceutical composition disclosed herein for the treatment of the patient's symptoms. One or more of the patient's symptoms generally stabilize.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of reversing accumulation of a serine protease in an organ of a subject, comprising:
(a) selecting a subject having or at risk of accumulation of a serine protease in the organ; and
(b) administering a therapeutically effective amount of a serine protease inhibitor,
thereby reversing accumulation of the serine protease in the organ of the subject.

2. A method of reversing cellular damage in an organ of a subject, comprising:
(a) selecting a subject having or at risk of cellular damage to the organ; and
(b) administering a therapeutically effective amount of a serine protease inhibitor,
thereby reversing cellular damage in the organ of the subject.

3. A method of preserving extracellular matrix in an organ of a subject, comprising:
(a) selecting a subject having or at risk of loss of extracellular matrix in the organ; and
(b) administering a therapeutically effective amount of a serine protease inhibitor,
thereby preserving extracellular matrix in the organ of the subject.

4. The method of any claim of the above, wherein the subject at least 40 years old.

5. The method of any claim of the above, wherein the subject at least 50 years old.

6. The method of any claim of the above, wherein the subject at least 60 years old.

7. The method of any claim of the above, wherein the subject is not at risk of developing shock and/or septic shock.

8. The method of any claim of the above, wherein the subject does not have HIV.

9. The method any claim of the above, wherein the organ selected from the group consisting of the brain, spinal cord, heart, kidney, muscle, liver, and lung.

10. The method any claim of the above, wherein the organ selected from the group consisting of the brain, heart, and muscle.

11. The method of any claim of the above, wherein the organ is the brain.

12. The method of any claim of the above, wherein selecting comprises selecting a subject with a brain disease or condition.

13. The method of claim 12, wherein the brain disease or condition is selected from the group consisting of Alzheimer's Disease, dementias including frontotemporal dementia, epilepsy or other seizure disorders, mental disorder, multiple sclerosis, Huntington's Disease, Parkinson's Disease, amyotrophic lateral sclerosis, meningitis, encephalitis, brain cancer, Crutzfeldt-Jakob disease, chronic traumatic encephalopathy, long-haul COVID associated dementia, and stroke.

14. The method of any claim of the above, wherein the organ is the heart.

15. The method of any claim of the above, wherein selecting a subject comprises selecting a subject with heart disease or a heart condition.

16. The method of claim 16, wherein the heart disease or condition is selected from the group consisting of coronary heart disease, angina, unstable angina, heart failure, cardiac arrhythmias, valve disease, high blood pressure, heart arrhythmias, endocarditis, pericardial disease, and cardiomyopathy.

17. The method of any claim of the above, wherein the organ is muscle.

18. The method of any claim of the above, wherein selecting a subject comprises selecting a subject with muscle disease or condition.

19. The method of claim 18, wherein the muscle disease or condition is selected from the group consisting of fibromyalgia, myositis, including polymyositis and dermatomyositis, muscular dystrophy, myasthenia gravis, amyotrophic lateral sclerosis, rhabdomyolysis, cardiomyopathy, sarcopenia, Charcot-Marie-Tooth disease, multiple sclerosis, myopathy, peripheral neuropathy, and spinal muscular atrophy.

20. The method of any claim of the above, wherein the organ is the kidney.

21. The method of any claim of the above, wherein selecting a subject at risk comprises selecting a subject with a kidney disease or condition.

22. The method of claim 21, wherein the kidney disease or condition is selected from the group consisting of chronic kidney disease, diabetic kidney disease, acute kidney injury, kidney stones, kidney infections, including pyelonephritis, kidney cysts, and kidney cancer.

23. The method of any claim of the above, wherein the organ is the liver.

24. The method of any claim of the above, wherein selecting a subject comprises selecting a subject with a liver disease or condition.

25. The method of claim 24, wherein the liver disease or condition is selected from the group consisting of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, and nonalcoholic steatohepatitis.

26. The method of any claim of the above, wherein the serine protease comprises at least one of a trypsin, a subtilisin, or combinations thereof.

27. The method of any claim of the above, wherein the serine protease comprises at least one of a trypsin, an elastase, a chymotrypsin, or combinations thereof.

28. The method of any claim of the above, wherein the serine protease comprises a trypsin.

29. The method of any claim of the above, wherein the serine protease inhibitor is a competitive inhibitor.

30. The method of any claim of the above, wherein the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin.

31. The method of any claim of the above, wherein the serine protease inhibitor comprises tranexamic acid.

32. The method of any claim of the above, wherein the therapeutically effective amount of the serine protease inhibitor is less than 10% of the subject's digestive enzyme activity.

33. The method of any claim of the above, wherein the therapeutically effective amount of the serine protease inhibitor is less than 10 μM.

34. The method of any claim of the above, wherein the therapeutically effective amount of the serine protease inhibitor is less than 5 μM.

35. The method of any claim of the above, wherein the serine protease inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump.

36. The method of any claim of the above, wherein the serine protease inhibitor is orally administered.

37. The method of any claim of the above, wherein the serine protease inhibitor is administered by a telemetry-controlled infusion pump.

38. The method of any claim of the above, wherein the serine protease inhibitor is administered as a liposome composition or as a nanoparticle encapsulation.

39. The method of any claim of the above, wherein the serine protease inhibitor is administered as an eye drop.

40. The method of any claim of the above, wherein the telemetry-controlled infusion pump is directed toward the organ.

41. The method of any claim of the above, wherein the serine protease inhibitor is administered for more than 1 week.

42. The method of any claim of the above, wherein the serine protease inhibitor is administered for more than 2 weeks.

43. The method of any claim of the above, wherein the serine protease inhibitor is administered for more than 4 weeks.

44. A pharmaceutical composition for the treatment of aging or age-related conditions comprising a serine protease inhibitor.

45. The pharmaceutical composition of any claim of the above, wherein the age-related condition affects an organ selected from the group consisting of the brain, spinal cord, heart, kidney, muscle, liver, and lung.

46. The pharmaceutical composition of any claim of the above, wherein the age-related condition affects an organ selected from the group consisting of the brain, heart, and muscle.

47. The pharmaceutical composition of any claim of the above, wherein the organ is the brain.

48. The pharmaceutical composition of any claim of the above, wherein the age-related condition is selected from the group consisting of Alzheimer's Disease, dementias including frontotemporal dementia, age-related loss of neuronal function, including but not limited to memory, balance, sensation, pain, epilepsy or other seizure disorders, mental disorder, multiple sclerosis, Huntington's Disease, Parkinson's Disease, amyotrophic lateral sclerosis meningitis, encephalitis, brain cancer, and transient ischemic strokes.

49. The pharmaceutical composition of any claim of the above, wherein the organ is the heart.

50. The pharmaceutical composition of any claim of the above, wherein the age-related condition is selected from the group consisting of coronary heart disease, angina, unstable angina, heart failure, valve disease high blood pressure, heart arrhythmias, endocarditis, pericardial disease, and cardiomyopathy.

51. The pharmaceutical composition of any claim of the above, wherein the organ is muscle.

52. The pharmaceutical composition of any claim of the above, wherein the age-related condition is selected from the group consisting of fibromyalgia, myositis, including polymyositis and dermatomyositis, muscular dystrophy, myasthenia gravis, amyotrophic lateral sclerosis, rhabdomyolysis, cardiomyopathy, sarcopenia, Charcot-Marie-Tooth disease, multiple sclerosis, myopathy, peripheral neuropathy, and spinal muscular atrophy.

53. The pharmaceutical composition of any claim of the above, wherein the organ is the kidney.

54. The pharmaceutical composition of any claim of the above, wherein the age-related condition is selected from the group consisting of acute kidney injury, kidney stones, kidney infections, including pyelonephritis, kidney cysts, the subject being in need of renal dialysis, and kidney cancer.

55. The pharmaceutical composition of any claim of the above, wherein the organ is the liver.

56. The pharmaceutical composition of any claim of the above, wherein the age-related condition is selected from the group consisting of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, and nonalcoholic steatohepatitis.

57. The pharmaceutical composition of any claim of the above, wherein the serine protease inhibitor is a competitive inhibitor.

58. The pharmaceutical composition of any claim of the above, wherein the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin.

59. The pharmaceutical composition of any claim of the above, wherein the serine protease inhibitor comprises tranexamic acid.

60. The pharmaceutical composition of any claim of the above, wherein the serine protease inhibitor is administered at less than 10% of the subject's digestive enzyme activity.

61. The pharmaceutical composition of any claim of the above, wherein the serine protease inhibitor is less than 10 μM.

62. The pharmaceutical composition of any claim of the above, wherein the serine protease inhibitor is less than 5 μM.

63. The pharmaceutical composition of any claim of the above, wherein the serine protease inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump.

64. The pharmaceutical composition of any claim of the above, wherein the serine protease inhibitor is orally administered.

65. The pharmaceutical composition of any claim of the above, wherein the serine protease inhibitor is administered by a telemetry-controlled infusion pump.

66. The pharmaceutical composition of any claim of the above, wherein the serine protease inhibitor is administered in as a liposome composition or a nanoparticle.

67. The pharmaceutical composition of any claim of the above, wherein the serine protease inhibitor is administered as an eye drop.

68. The pharmaceutical composition of any claim of the above, wherein the telemetry-controlled infusion pump is directed toward the organ.

69. The pharmaceutical composition of any claim of the above, wherein the serine protease inhibitor is administered for more than 1 week.

70. The pharmaceutical composition of any claim of the above, wherein the serine protease inhibitor is administered for more than 2 weeks.

71. The pharmaceutical composition of any claim of the above. wherein the serine protease inhibitor is administered for more than 4 weeks.
